(19) United States
(12) Patent Application Publication
(10) Pub. No.: US 2009/0211585 A1
(43) Pub. Date: Aug. 27, 2009

(54) Coverings for Phototherapy and Topical Treatment

(76) Inventors: William E. Cumbie, Yorktown, VA (US); Douglas B. Juanarena, Blacksburg, VA (US)

Correspondence Address: MEREK, BLACKMON & VOORHEES, LLC, 673 S. WASHINGTON ST., ALEXANDRIA, VA, 22314, US

(21) Appl. No.: 12/130,085

(22) Filed: May 30, 2008

Related U.S. Application Data
(60) Provisional application No. 60/940,712, filed on May 30, 2007.

(51) Int. Cl. A61B 19/08; A43B 17/00

(52) U.S. Cl. 128/849; 2/239

(57) ABSTRACT

A method to effectively cover parts of the body that should not receive phototherapy treatment. The invention can also be used to cover parts of the body that are not receiving topical treatments and are adjacent to such areas being treated. Three variations of the covering device can be used separately or together in various combinations. An extremity covering device covers a large portion of a hand, foot or other extremity. A digit covering device covers one finger or toe. A skin covering device is used to cover areas not covered by the extremity or digit covering devices, or can be used separately to cover a finite area of the skin, nails, or other portion of a patient.

### CROSS REFERENCE TO PRIOR APPLICATION

This application claims the benefit of U.S. Provisional Application Ser. No. 60/940,712, filed May 30, 2007.

### BACKGROUND—FIELD OF INVENTION

This invention relates to phototherapy and a means to selectively expose treatment areas or mask areas of the body not being treated by phototherapy. The coverings can also be used for topical applications and treatments.

### BACKGROUND OF THE INVENTION—OBJECTS AND ADVANTAGES

Phototherapy is used to treat a large number of disorders and conditions. However, during phototherapy it is often desirable to selectively treat one or more areas while avoid accidental application of the treatment to areas not being treated by masking or protecting areas of the body not being treated. The present invention provides a way to effectively cover the parts of the body that are not to receive phototherapy treatment. The invention can also be used to cover parts of the body that are not receiving topical treatments and are adjacent to such areas being treated. The invention has the following advantages compared with trying to cover an area without a system:

Firstly, the covering system can greatly reduce the time necessary to cover an area by providing a covering designed for the intended purpose that may readily and quickly applied.

Secondly, the covering system can make use of preselected, tested materials that have been shown to cover an area well and are impervious to the treatment to prevent exposure to areas to areas that are not being treated. Different materials may be designed or used for different types of treatment, such as UV filtering materials or chemical and drug resistant materials.

Thirdly, use of the present covering system can ensure proper, approved masking materials are used, thereby preventing the undesired use of third party, non-preferred materials which either do not cover properly or which do not offer sufficient protection.

### SUMMARY OF INVENTION

One aspect of the invention is a method and device to rapidly and effectively cover areas of the body that are adjacent to areas being treated which need to be shielded from the treatment being applied. This is especially relevant when the side effects of the treatment could potentially be hazardous or when the area can generate a tolerance for the drugs or treatment making later treatment of that area more difficult.

It is therefore an object of the invention to provide a system comprising a number of discrete types of coverings that may be used independently or together to control the application of treatment to an individual. The treatment may be a phototherapy treatment or a topical medication, by way of example.

It is another object to provide a cover for a patient that has an opening for allowing treatment access to a particular area while screening or masking the surrounding areas.

It is a further object of the invention to provide a covering that is impervious to light, UV light, medicaments or other intended treatment(s).

It is yet another object of the invention to provide a covering system that can be used together in various combinations to efficiently cover large and small areas of skin or nails of a user.

It is an additional object of the invention to provide a cover that mates with a treatment device to prevent light from escaping from the treatment device past the covering.

These and other objects of the present invention will become apparent upon review of the following specification and drawings. Not all of the objects of the invention need be present in each embodiment of the invention.

### BRIEF DESCRIPTION OF DRAWINGS

FIG. 1 shows an exploded view of the three components of the covering system. FIG. 2 shows an ECD (extremity covering device).

FIG. 3 is the DCD (digit covering device) after it has been applied to a large toe that will have its nail treated

FIG. 4 is the SCD (skin covering device) with several typical variations in shape. This figure also shows the SCD after it is applied around the skin of the nail of a large toe and a small toe that will have its nail treated.

FIGS. 5A and 5B are SCD (skin covering device) variations. These are illustrative, not exhaustive. The shape of the SCD may be either an arc, a semicircle, a ‘U’ shape, a ‘horseshoe’, a complex curve, or a combination thereof.

FIG. 6 shows a two part system where the DCD has been integrated into the SCD and the ECD. The SCD is first applied over the toes to be treated (large toe and small toe) and then the ECD is also shown after the end covering the large and small to have been cut off and applied to the foot. The area around the nail is now fully covered and the nail is ready for treatment.

FIG. 7 is an ECD that can be fitted to a device used to treat disorders.

FIGS. 8A & 8B show an ECD fitted to a device used to treat disorders where the ECD has a flange that can be easily mated to its complementary opening in the device.

FIG. 9 is a graphic of an integrated covering system that has tabs that can removed to uncover the area to be treated

FIG. 10 is a covering that can be trimmed to expose the area to be treated. In this figure the covering that has been modified by scoring and by ridges to assist in the trimming of material and to prevent tears from propagating.

FIG. 11 is one example of the device to cut or trim material away from the area to be treated. (Precision depth knife).

FIG. 12 is a sketch of a method using a template to cut out the correctly sized opening to expose the area to be treated.

FIG. 13 is a diagrammatic view of application of a system having only an ECD and SCD to totally cover the extremity being treated.

FIG. 14 shows an ECD having a flange for mating with a phototherapy device.

### DETAILED DESCRIPTION OF INVENTION

New treatments of skin and nails and other portions of the human body have been developed that can potentially cure previously incurable diseases and other maladies. Some of these treatments, while promising exciting cures, may have potentially damaging side effects. It is important thus to provide these treatments, such as phototherapy and application of topical agents, only to the intended area to limit these side effects. Additionally, some types or quantities therapies may be suitable for one portion of a patient, such as a finger nail or toe nail, and not another surrounding area such as the skin. Alternatively, it may be important to apply different types or quantities of treatment to various portions of a patient.

It is often therefore important to be able to apply treatments to a particular area while covering or masking adjacent areas to the area being treated by, for example, phototherapy or by topical therapy to control the application of a therapy. One aspect of the invention is a method and means to cover or mask areas adjacent to areas being treated that are not to be treated during a phototherapy or topical therapy. Alternatively, an entire portion of a patient may be covered with a mask, and subsequently a portion of the mask covering the area to be treated may be removed to expose the intended treatment area. Of particular interest, an area to be masked, may be contained on the extremities of the body such as the hands and feet, where it may be desirable to selectively mask the nails or skin.

The invention comprises a method to protect non-treatment areas from exposure to phototherapy or topical treatment, especially when the non-treatment areas are adjacent to areas to be treated. Various methods are described which take into account conformational differences of diverse parts of the body.

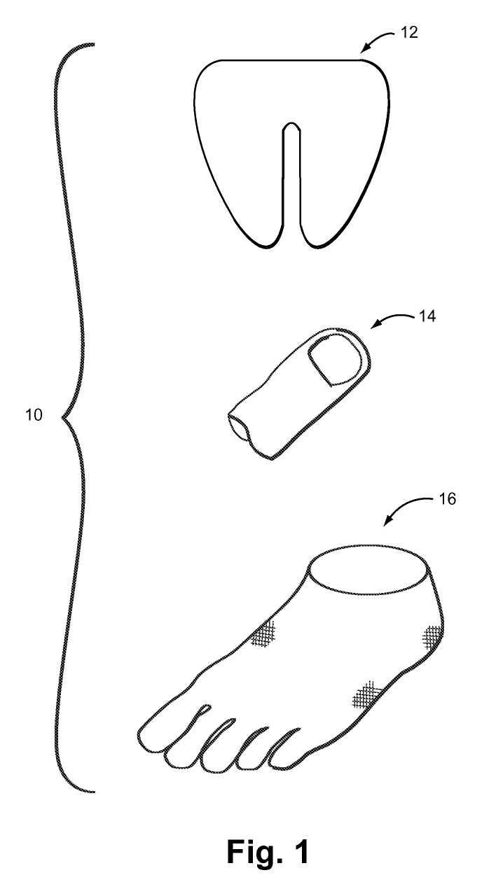

Three preferred devices are contemplated by invention which may be usable separately or together in any combination to cover the skin or nail or a user include: 1) an Extremity Covering Device (ECD), namely a covering for the foot or hand as a whole or covering a major portion, 2) a Digit Covering Device (DCD), namely a secondary covering for the digit(s) to be treated, and 3) a Skin Covering Device (SCD), namely a tertiary covering to mask the edge of the skin immediately adjacent to the nail. These parts are functional parts and one or more may be combined to form any single physical unit. Moreover, if the therapy is applied in a manner that it does not cover a large area but is instead concentrated in one area, such as a light that has a one-inch diameter circular treatment area or a brush applying, the first piece of the system (the ECD) or the second part of the system (the DCD) may not be necessary. Also, in some circumstances, the extremity covering device may not be required if the area to be treated is not located on an extremity. Examples of these parts are shown in FIG. 1.

FIG. 1 shows the three components of the covering system 10, namely the SCD (skin covering device) 12, the DCD (digit covering device) 14, and the ECD (extremity covering device) 16 that may be used separately or together to mask or uncovered preferred areas. Additionally, one or more of these components may be combined into one physical unit. These devices may be applied in various orders, as necessary, to quickly and easily mask or unmask an area. It is not necessary to use each device for each application. For example, a DCD may not be necessary if a digit is not to be individually covered. The ECD may not be necessary if an extremity is not covered.

FIG. 2 shows an Extremity Covering Device (“ECD”) 16. This first piece of the system is a covering for the foot or hand (not shown) and is similar to a toe sock, bootie or a glove, including a heel portion 18 and a toe portion 20. The ECD may coverg the entire foot, however, it need only be comprised of the individual coverings for each toe and approximately 1-inch of material extending back along the foot, measured from where the toes begin, so that it can join these coverings together. In practice, a foot is inserted into the ESD through an opening 22 in the back or top of the device until the toes extend to the end of the covering. It is also envisioned that the device could also be similar to a slipper except that it had toes also if treatment would not extend to the heel portion. Or the device could just cover the end of the foot with toes (i.e. the covering does not extend back to the ankle or heel). This piece is designed to provide comfort during treatment and to block or appropriately filter light during treatment. The device is capable of being easily modified to expose the nail(s) or other area(s) to be treated. This piece provides comfort, ventilation if worn for a long time, and cooling as well as shielding of the light. This piece is necessary only if there is an area on an extremity that needs to be treated. An example of this part is shown in FIG. 2. While FIG. 2 shows the ECD covering the entire foot the covering need only extend approximately 1-inch back towards the heel from where the toes begin so that the covering comprises at least the coverings for the individual toes and enough material so that it can hold the toes coverings together.

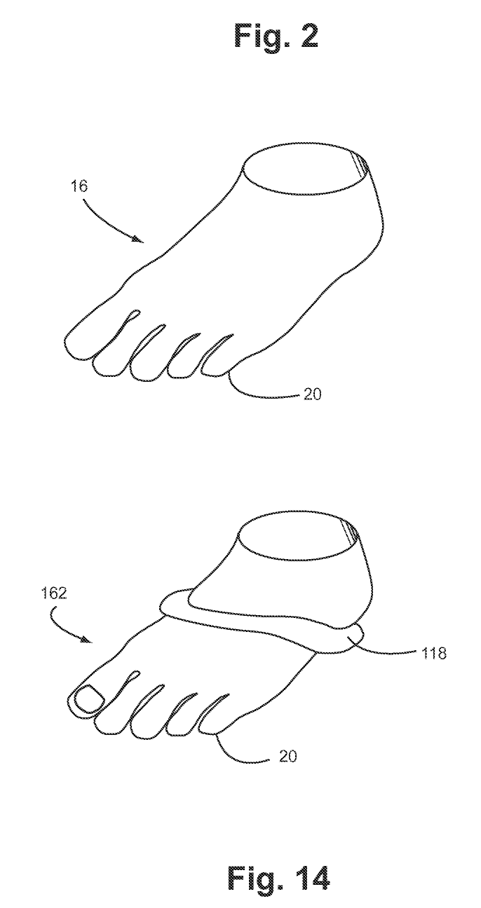

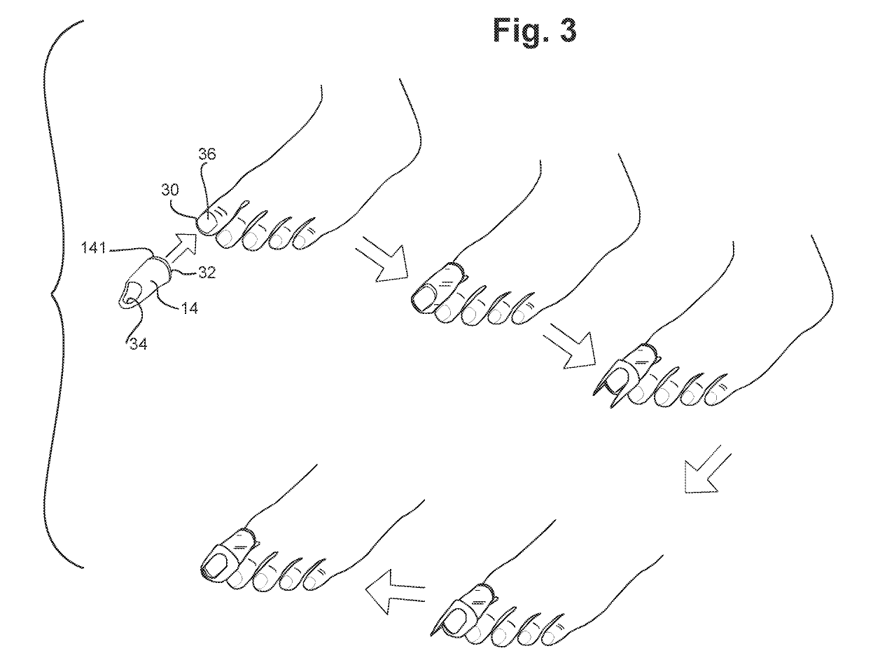

FIG. 3 shows a Digit Covering Device (“DCD”) 14, also called a Digital Covering Unit (“DCU”). The DCD may have many various sizes and shapes, but in a preferred embodiment shown in FIG. 3 is sized to fit one toe 30. An opening 32 provided at a first end receives a tow within. A second opening 34 is provided for exposing an area of the toe for treatment. In the example shown in FIG. 3, an opening 32 exposes the nail 36 of the toe 30 so that a treatment such as phototherapy can be applied.

The DCD of the system is similar to a finger cot but comes in a variety of sizes permitting the correct size to be selected for the digit 30 being treated. This piece is designed to both provide comfort during treatment and total blocking of light during treatment to areas covered by the DCD. The DCD can also be used to provide ventilation and cooling as necessary. One preferred variant of the DCD is for the DCD to include connection to a external or integral unit to permit cool or warm air through to be circulated through the device to adjust the temperature of the digit to provide optimal treatment and to provide additional comfort. For example the DCD could be used to heat an area being treated in order to stress the organisms causing the infection, thus improving the efficacy of the treatment. Conversely, if such heat was not required for treatment it could be used to cool the area being treated to make the treatment more comfortable. Although the DCD is the second piece in the total system 10, it is contemplated that it may be the first that is applied to the hand or foot being treated or it may be second after the SCD. The DCD will be fitted to the subject based on the size of the digit being treated. This piece has a fixed or variable cutout 34 on top that will permit the nail 36 to be exposed during treatment. The DCD covers most of the skin around the nail to be treated. However, it is preferably designed to be used with a third piece, the Skin Covering Device (“SCD”) 12, which is an adhesive masking system that is designed to cover small amounts of skin or surface that remains uncovered by the digit covering unit. The digit covering device may comprise something similar to a finger cot with a elastomeric exterior and may include a lined interior (such as cotton or flocking). It may have a positive means to permit it to stay in place, such as, by way of example, an interior adhesive, an adhesive strip placed around its circumference and the skin, a non-slip interior surface, or by friction fit, etc. The SCD can be precut or it can be custom cut to fit around a nail.

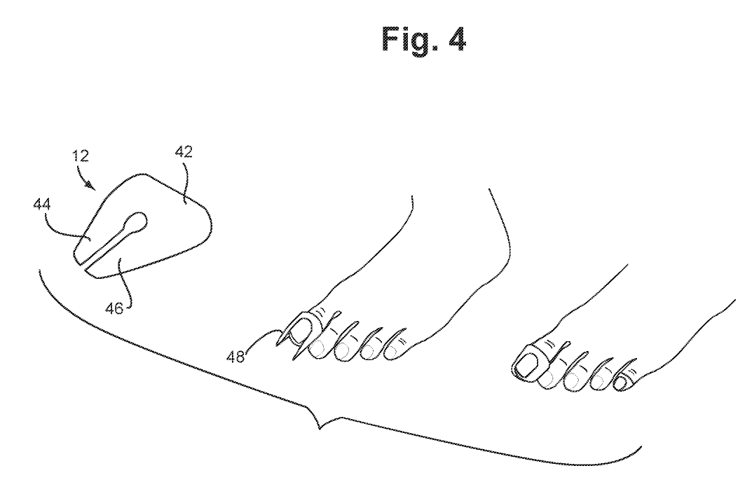

FIG. 4 shows an example of a Skin Covering Device (SCD) 12. The SCD may be a third piece of the overall covering system. The SCD is a skin masking unit which permits the masking of the small amount of skin exposed between the DCD and the nail to be treated. This allows for more accurate positioning of the covering to fill gaps exposed by or not covered by the other units. The SCD 12 comprises a base 42 and two legs 44,46 generally forming a U shape with a void formed between the three portions. In practice, the base is applied to an extremity or digit such as a toe. To treat a nail, the base would be applied along the base of the nail with the two legs 44,46 extending along the sides of the nail. The ends of the legs 44,46 may then be crossed over each other at the tip of the nail and toe to completely encircle the nail 48. The SCD preferably contains its own adhesive on one surface of the SCD to accurately position the SCD on the toe and maintain the SCD in the preferred space. However, it is also contemplated that external adhesives or tapes or the like could be use to hold the SCD in place.

This SCD preferably comprises a material or layer that can shield the skin from harmful light or topical medicaments or other compounds used during treatment. Like the DCD, the SCD can remain in place after treatment and serve as a mask and holder for application of a topical dressing to the nail which can further treat nail infections. For example, a sorbent pad of fungicide may be applied to the nail after treatment to act synergistically with the phototherapy treatment.

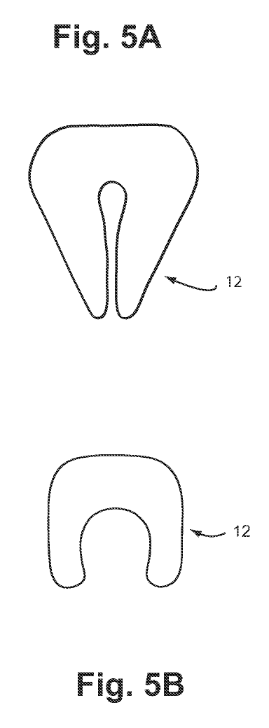

The SCD can be specially designed to be easily applied to mask off the area being treated. For example it can be curved in an arc (such as a ‘U’ shape, a horseshoe shape, a circular shape, etc.) so that it can be applied to a roughly circular area such as what would be needed to treat a nail or a wart, etc. The SCD can also be specially formulated with a light adhesive that keep the unit in place but is also easily removed without pulling the skin or hair on the skin. An example of the SCD applied around toe nails is shown in FIG. 4. FIGS. 5A and 5B show some representative variations on an SCD 12 to cover areas.

The SCD can also be used to contain a material that aids in penetration of the light such as an optical clearing liquid which can enhance transmissivity (such using glycerin which may inhibit scattering of the light). In this use the SCD can create a small well wherein the fluid can be added.

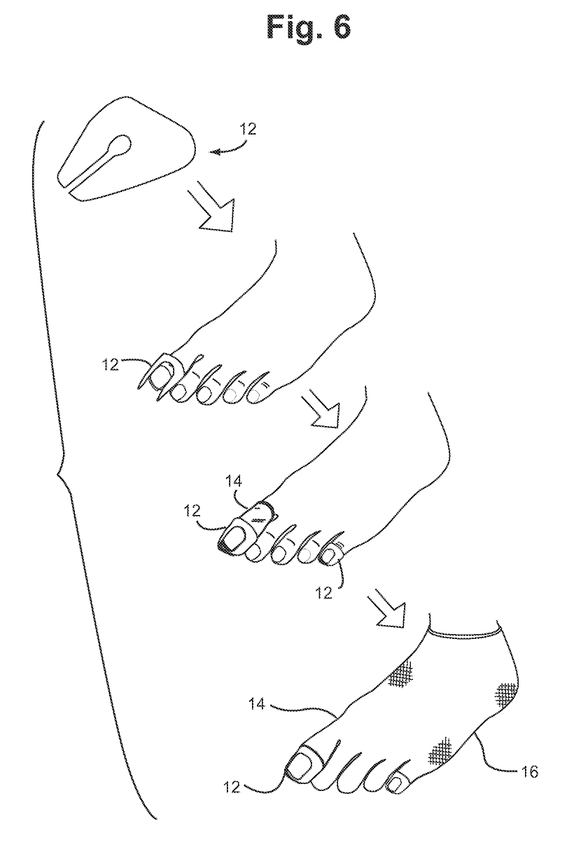

The three pieces that comprise the system, namely the ECD, DCD and SCD may be used separately or two or all three of them may be incorporated into one unit. In particular an ECD and SCD may be used to form a complete covering if they are matched to meet or overlap each other so that fine tuning using the DCD is not necessary. An example of a two piece system is shown in FIG. 6.

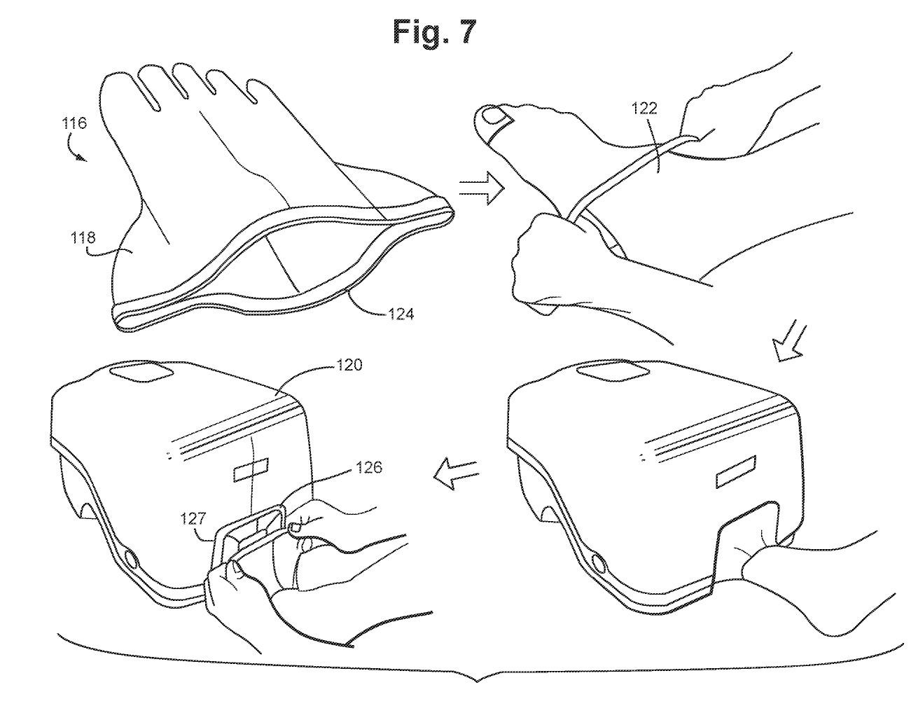

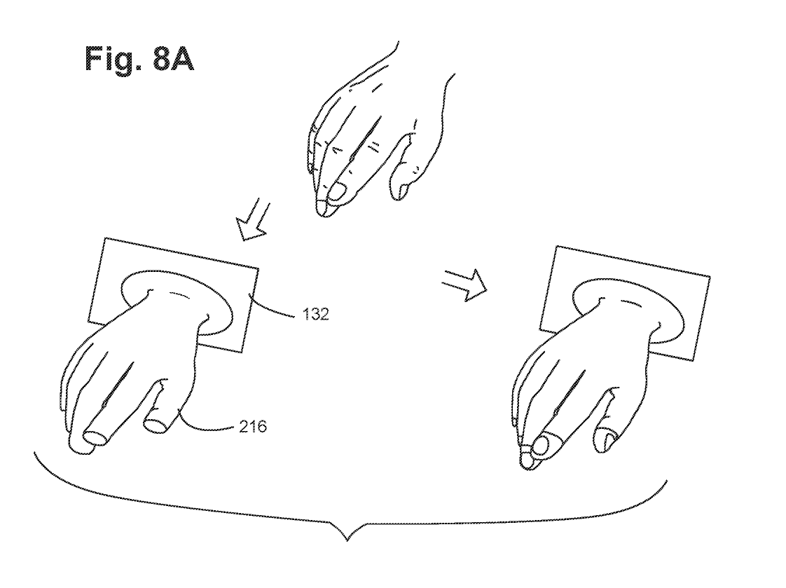

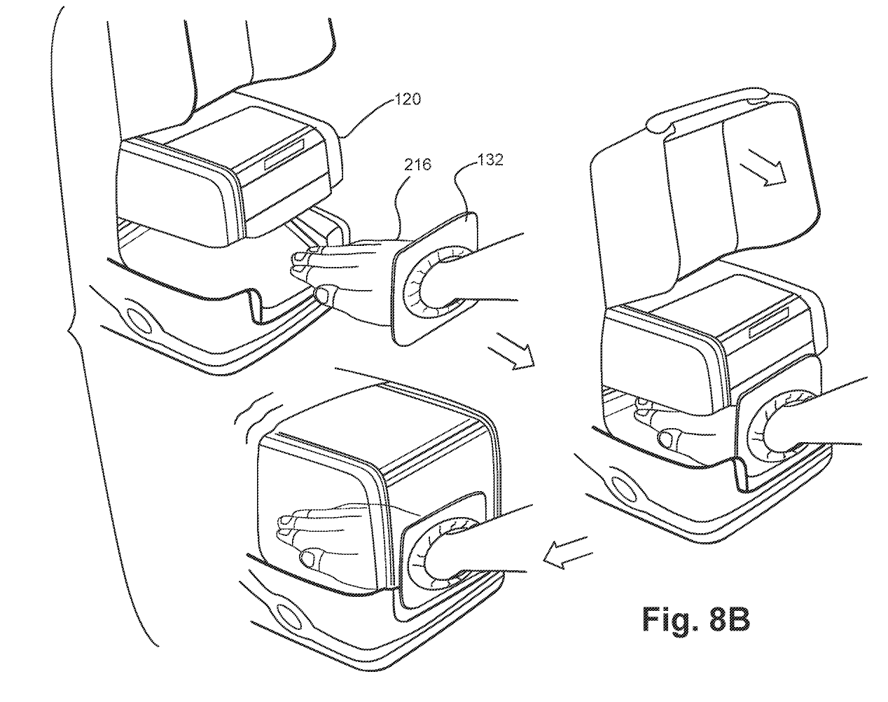

Additionally, the covering system may be integrated to specifically mate with the device used to treat a disorder. FIGS. 7 and 8 illustrate two ways that the ECD can be integrated into a device to treat a disorder. FIG. 7 shows an ECD 116 designed to mate with a treatment device such as a phototherapy unit 120. In practice the ECD is installed over a portion of a user such as a foot 122. A light adhesive may be used to maintain the ECD in place on the foot, but preferably is held in place by friction. Alternatively, the ECD 162 portion installed over the foot may extend back to the heel either by having a heel portion (see FIG. 14) or may have a strap portion (not shown) extending around or securable behind the heel by various methods including buckles, elastic straps, hook and loop fasteners, hooks, buttons, etc. In FIG. 7, the ECD portion is shown extending only to around mid foot, however.

A flange portion 118 of the device 116 may be folded back over a portion of the device to connect to the phototherapy unit 120 to form a seal with the unit to completely block any light from escaping the unit. A band 124 may be formed of elastic to mate with a reverse tapered receiver flange 126. The receiver flange 126 may have a narrow neck portion 127 so that the elastic band 124 may be secured around the neck portion to prevent the band from slipping past the larger flange portion 128. Other mating devices such as hook and loops, hooks, adhesive, etc., may be alternatively used to connect the ECD to the phototherapy device.

FIGS. 8A & 8B show a variation of the mating ECD and phototherapy device. In this example, a flange 132 is provided on the ECD 216. The flange may be partially flaccid or may be rigid to maintain its own shape. However, it is preferred that the flange is held in place by connecting to a flange or receiver on the treatment device 120 by connectors, air pressure (“suction”), adhesives, hook and loop fasteners or the like. The flange connected to the ECD could have many shapes including square, rectangular, oval, circular, or irregular. The vertical distance from one end of the flange to the other could vary from 3-inches to 6-inches while the horizontal distance end to end could vary from 3-inches to 6-inches. If only one digit was being treated the flange would attach to a DCD and would have a maximum flange size of 3-inches end to end both vertically and horizontally. The single digit flange unit would be particularly suited to treatment of fingers.

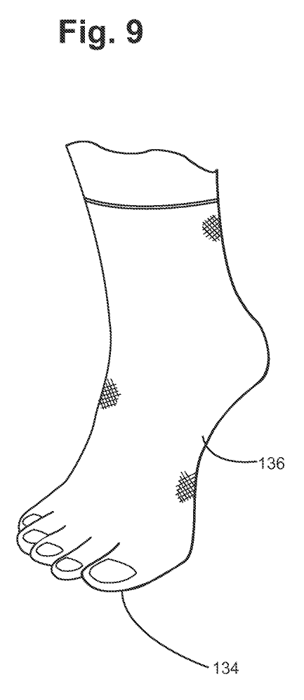

The covering system could be integrated into one unit 136 such as shown in FIG. 9 where the covering has tabs 134 or sections that can be removed to expose the nails to be treated. In this case either all parts could be integrated or it may be that the SCD would still need to be applied if a tab were removed and too much skin was exposed next to the nail to be treated. The system may comprises items that need to be cut or trimmed to expose the area to be treated while covering the area that is not to be treated.

The system may be combined into one unit that would fit over a hand or foot like a glove, mitten, toe sock, or regular sock. The system could have be formed of a stretchable material to permit one item to fit a number of different hand or foot sizes. In this manner it may be necessary to have only one size or a small number of sizes necessary to treat a wide range of people. The system could be put on the extremity and the area to be treated could be exposed by cutting or trimming away material that covers the area to be treated. The system could be made of a material that could be trimmed easily but would have good resistance to tearing once it was trimmed. Score lines, perforations, or natural rip lines could be used to aid in tearing and to ensure that that only the intended area was removed. Templates, guides or ridges could be used to aid in identifying the areas to be cut ruing marking or cutting of the material.

Each portion of the system could be made of any material that will sufficiently filter, block or resist the treatment from reaching the areas that are not to be treated including rubber, latex, nitrile, silicone, other elastomers and plastics, foil, cloth, clay, and similar materials. The materials could be treated or layered to aid their natural properties in the resistance to treatment. Some materials such as vinyl are not naturally UV opaque but can be made so by adding substances to change their characteristics. One such substance to add UV blocking to a material is the addition of a UV blocking substance such as carbon black which is effective even when added in quantities of less than 1% by weight. Coating the inside (perhaps by adding flocking) or the outside (using a lightly sprayed coating) are also ways to increase UV blocking. Preferably the UV blocking material is nearly opaque to UV light, but could be SPF 15 or greater, for example, SPF 30, SPF 45 or higher.

Of the materials that can be used for coverings natural rubber, latex, nitrile, silicone, and neoprene are the preferred materials due to their ability to filter out or block UV, stretch and conform to shapes, and their tear resistance. Of these latex would be the material of choice for many applications, however, some people exhibit allergies to latex which makes the use of nitrile, silicone and neoprene also viable choices as they tend to be less allergy prone. Thicknesses of 3 mils or more are preferable unless additional UV blocking material such as carbon black or titanium dioxide has been added. Preferable thickness is in the range of 4 mils to 10 mils for a covering that will be disposable. Greater thicknesses may be selected if the covering will be used more than once or for to make it more comfortable. Polyurethane, polyethylene (all types—ldpe, xdpe, hdpe, etc.) and vinyl are less preferred alternatives that can be improved on by enhancing their UV blocking ability and their ability to stretch.

Covering material used in UV phototherapy should filter or block at least 99.5% of all UV light from the phototherapy treatment. The covering may, for example, be made of a UV blocking material, may have a UV blocking material added, or may be coated with a UV blocking coating. Preferably, the covering material used in UV phototherapy should filter or block at least 99.9% of light between 240 nm and 320 nm. There are several ways to ensure that the covering is acceptably opaque to UV. One is to use materials with known UV filtering and blocking characteristics and select a thickness of the material such that it will filter or block the required amount of UV when used in this application. Using this approach the covering should be constructed with a sufficient factor of safety to take into account any variability in the material. For example, if a certain formulation of nitrile can blow 99% of all UV and 99.5% of UV between 240 nm and 320 nm when it is 3 mils thick and the variability of the extrusion process is +/−10% then the material should be at least 3.3. mils thick. Additionally, if it is calculated that the silicone may become thinner due to stretching this would also need to be taken into account. For example if the nitrile would become 20% thinner when stretch by putting it on a foot then the thickness should be likewise increase by at least 20%. In this case the 3.3 mils would then become 3.96 mils or rounded up to 4 mils. An added factor of safety may also be added to ensure that sufficient UV is filtered or blocked, especially if no or few actual coverings are tested after fabrication to ensure they do indeed filter or block the requisite UV. Another way to ensure that sufficient UV is blocked is to test a statistically significant number of coverings and make sure they meet the requirements. A final approach is to test every covering to ensure it meets the requirements.

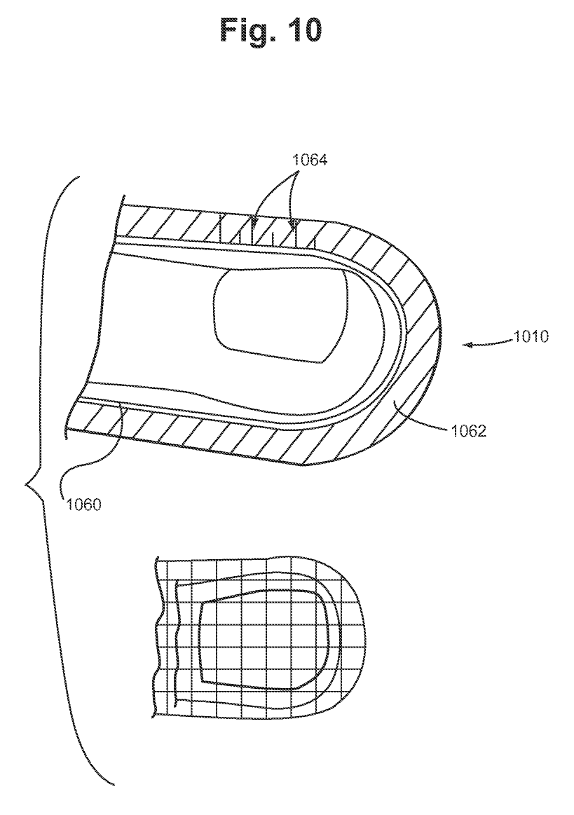

Component of the filtering or blocking system could also make use of an amorphous material such as clay or an ointment (for example zinc oxide squeezed out of a tube) to also mask part of an area. The system could be transparent or translucent or clear to facilitate trimming or cutting in the correct area as shown in FIG. 10. The system 1010 could be composed of a composite material such as a layer of silicone 1060 next to the skin with a layer of latex 1062 on top which could be scored 1064 to permit easy cutting away of material and also offer good tear resistance when cut. These examples are illustrative and not definitive.

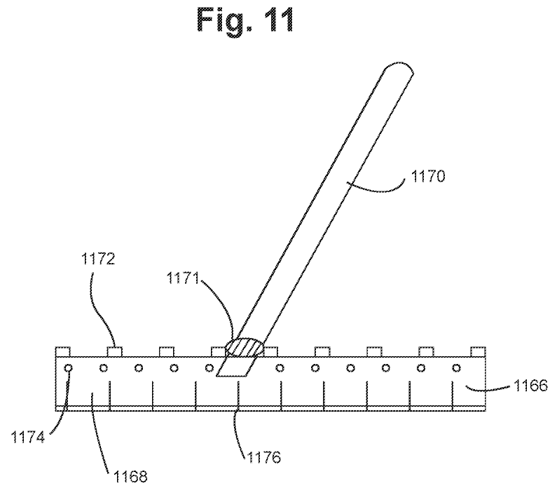

The material selected could be modified by means that would permit it to be cut easily while preventing tearing as shown in FIG. 11. For example, if an elastomeric material 1166 such as silicone was used and it required a thickness of 2 mm to filter or block sufficient light then the covering could be made to be 6 mm thick but have a scored undersurface 1168 where the scored are 3 mm depth and are located horizontally and vertically every 2 mm center to center with the scores being 0.5 mm wide. This would permit a precision cutting knife 1170 or the like that can penetrate to a depth of 3 mm to cut away the area to be exposed. A stop or flange 1171 may be provided to limit the depth of the cut. Since the total thickness is 6 mm, the knife would not touch the skin while the covering is being cut. Also, the covering could be similarly reinforced at points to prevent tears from propagating. This could be done by adding ridges 1172 to the covering. For example, the ridges can be similar to the score marks discussed earlier except they would extend beyond the surface and reinforce it. The reinforcing could also be a gridded mesh 1174 in two dimensions made of a transparent plastic such as monofilament. These examples are only illustrative and many similar modifications can be made and are claimed as part of this invention. This means of reinforcing and weakening a covering to make it easier to cut is specifically envisioned as a unique and unobvious method and can be applied to coverings and other items which require trimming and cutting. The adjunct precision depth cutting device that works in tandem with the scoring is also specifically claimed as a unique and unobvious invention. A sketch of a trimming device is shown in FIG. 11.

The coverings can be coated with an adhesive 1176 to help them remain in place during treatment. They may also be made of a material that is tacky to prevent slippage. The adhesive could be specially designed to adhere to moist skin or it could be designed not to cling to hair.

The DCD 14 (FIG. 3) can be made long so that it can be trimmed (short for small digits like small toes or it could be left long to treat longer digits such as a small fingernail). Conversely, if the DCD is made of a material that can stretch it can be made short and will stretch when it is applied to longer digits. The DCD may be provide with a flange 141, roll of material, straps or other devices that may be grasped to aid in pulling the DCD over the digit. The DCD may have portions that are opaque to light and portions of material that are transparent to the treatment light to define the area to be treated with or without cutting material away.

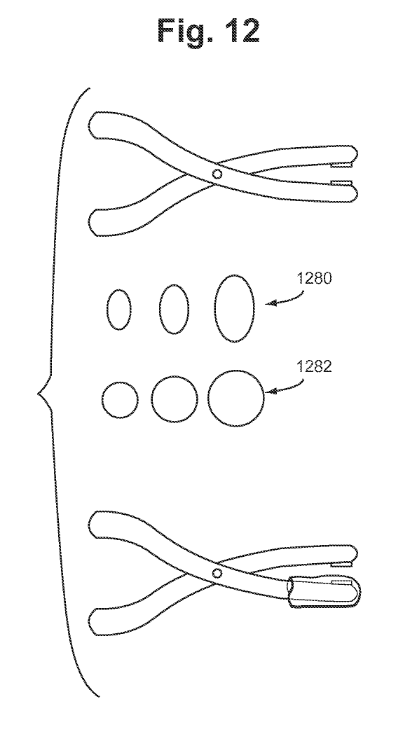

The DCD 14 may not have the nail opening precut but may instead be cut to better match the particular nail opening 34 (using a device similar a hole punch that can be fitted with dies that will cut out different size openings or other means that accomplish this end). See FIG. 12. An intermediate template 1280, such as paper, could be used to measure the area to be exposed. The template could be laid over the DCD 14 (FIG. 3) to cut the DCD without requiring the user's finger or toe be inside the DCD while it is being cut. Likewise, the covering could also have a small transparent material 1282 that you could trace the area being treated and then apply the traced area over a covering to use as a template to cut out an opening.

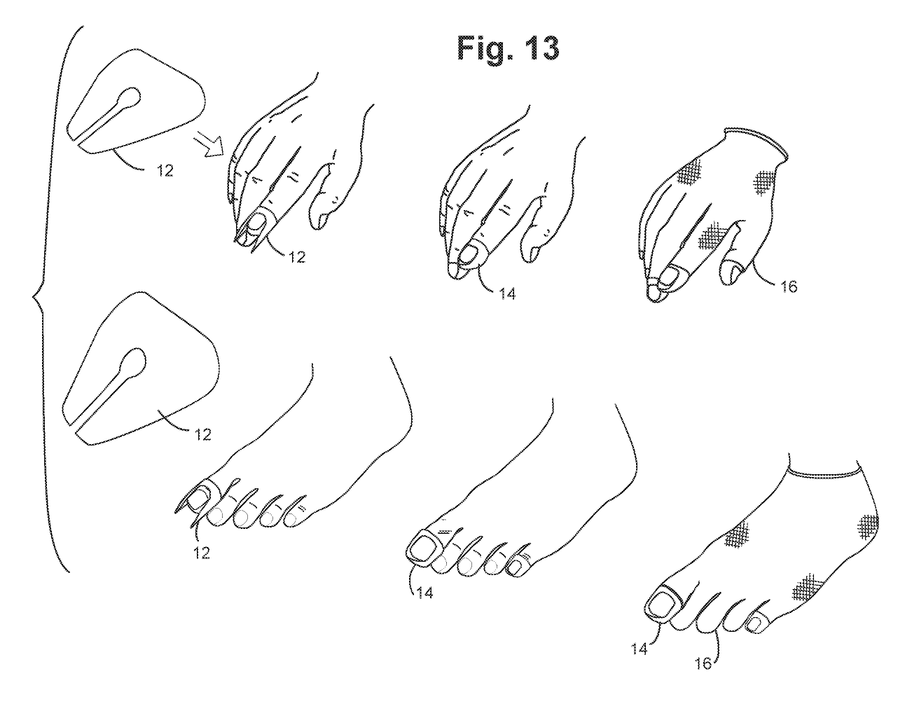

In practice, the system may be used to cover non-treatment areas on a user by first selecting and applying the DCD as shown in FIG. 13. Then selecting and modifying as necessary the ECD and put it on the extremity that has the nail to be treated. Finally, applying the SCD around the nail to be treated to minimize the amount of skin exposed to treatment. Alternatively, one may first apply the SCD, then apply the DCD over it, and finally apply the ECD. This latter sequence permits the skin to be covered first by a SCD that is wide enough to cover approximately ¼-in all around the nail. An SCD this wide permits the use of a smaller number of DCD sizes since the holes can be considerably larger than the nail opening itself

If the system combines the three devices into a single item, then the items will be put on a one time. However, it may be necessary to modify an item by repositioning it or by trimming or cutting parts to ensure it covers the necessary areas while exposing areas to be treated.

Other methods of applying the system are discussed in this application and are descriptive and not intended as a definitive listing if all ways to apply coverings for treatment.

Although the descriptions above contain many specificities, these should not be construed as limiting the scope of the invention but merely as providing illustrations of some of the presently preferred embodiments of this invention.

### DESCRIPTION OF PREFERRED EMBODIMENTS

In a preferred embodiment the DCD is not required because the ECD and SCD are designed to closely overlap and render the DCD unnecessary. In the preferred embodiment the ECD is made of a nitrile material that is dipped using a foot mold that is approximately the size of a male size 9 foot. The elasticity of the material permits subjects with larger feet to use the foot glove and those with smaller feet can use it although it may be loose in areas. An alternate preferred embodiment has several different size foot gloves to choose from for a better fit. The dipped product produces a foot glove that is similar in fit to a toe sock but ends below the ankle. The thickness of the device is preferably 10 to 15 mils for comfort, tear resistance, and UV blocking ability, and a medium durometer material is used. A thinner thickness of nitrile may be used with a thickness of 5 mils providing adequate UV filtering or blocking ability. An even thinner thickness could be used if a UV blocking agent such as carbon black (less than 1% by weight is acceptable) was added. An example of the preferred DCD embodiment is shown in FIG. 3. In the preferred embodiment the SCD is made of a medical grade foam tape which blocks UV. The foam tape 1201, 1202 is configured in an arc (roughly ‘horseshoe’ shape) as shown in FIGS. 5A and B. In the preferred embodiment the SCD is applied first around all nails that are to be treated. After the SCD is applied, the DCD is modified by cutting the tips off of the corresponding digit coverings and have the SCD applied to them. After this the DCD is slipped on to the foot of the nails that are receiving treatment. Alternatively, the DCD can be applied and the SCD applied to mask any non-treatment areas not covered by the DCD.

In another preferred embodiment the three components, the ECD, the DCD, and the SCD, are combined into one unit. The unit is similar in shape to the area to be treated and its surrounding area. For example the covering to expose one or more nails on a foot to treated while protecting the surrounding skin may look like a cross between a toe sock and a slipper (i.e. a slipper with toes). The material of construction must be able to block all unwanted treatment from the areas not to be treated. For example a covering similar to a toe sock may not be acceptable if the material of construction cannot prevent substantially all harmful light from reaching the area that is to be protected. In that case the covering would need to be modified suitably perhaps by applying an elastomer covering over the toe sock that could block light. In the preferred embodiment the covering would be one piece that is able to stretch and thus be useful to cover the extremity of a number of people of different sizes. For example the cross between a toe-sock and a slipper could be made of a material so that three sizes (small, medium, and large) would be able to cover substantially all people feet (although it may require a separate type of covering for the right and for the left foot). In the preferred embodiment the covering would be one piece that would cover the extremity but also show the area underneath that need to be exposed for treatment. This could be accomplished by using translucent materials or materials that are optically clear in the light ranges that will not harm the skin (i.e. it will filter out UV but not all visible light wavelengths). In the preferred embodiment the covering will be easy to trim or cut away and expose the area to be treated but will not tear easily. This may require a composite material such as using silicone (which cuts easily but also tears) next to the skin with a top coating of a material that is harder to tear such as latex or perhaps a webbed material. The top coating could be applied over all of the covering or just in the area that is normally adjacent the area cut away for treatment (in this case that would be the nails). The preferred embodiment could also have a specially designed device to cut or trim the material while not cutting the skin. Therefore, the currently preferred embodiment of the invention would be a covering that incorporates the ECD, the DCD, and the SCD, in one item that is made of a translucent elastomeric material that can conform and that had good tear resistance but which can be trimmed easily to expose the area to be treated along with a device to cut or trim away the unwanted material and which has been modified around the nail areas by scoring on the underside to permit easy trimming. The preferred embodiment is conceptually illustrated in FIG. 6.

In a preferred embodiment for shielding around a nail that will be treated, a three piece system comprise 1) the Extremity Covering Device (ECD)—a covering for the foot or hand as a whole, 2) the Digit Covering Device (DCD)—a secondary covering for the digit(s) to be treated, and 3) the Skin Covering Device (SCD)—a tertiary covering to mask the edge of the skin immediately adjacent to the nail.

ECD—Extremity Covering Device—The first piece of the system in the preferred embodiment is a covering for the foot or hand and is similar to a toe sock or a glove and is capable of blocking substantially all of the light during phototherapy. The ECD provides comfort, ventilation, and temperature control during treatment. In a preferred embodiment the ECD could be made of an elastomeric material that can easily conform to different size feet or hands. A variant of this preferred embodiment would use flocking on the inside surface of the elastomer. In another preferred embodiment when treating a toe nail the ECD may be a covering that is conformational to the foot (similar to a toe sock) with closely woven fabric that is approximately ¼-in thick or more. In the preferred embodiment that fabric would be breathable such as cotton and would also be stretchable—perhaps a blend of natural and synthetic materials—and that would also be hypoallergenic. The ECD in the preferred embodiment would have a secondary covering to further block light during treatment such as a coating of latex or other elastomer that can block substantially all light—especially that in the UV range. The secondary covering would have the capability to ‘breathe’ (such as canvas or flocking) or would not totally cover the entire foot to permit ventilation (for example, the bottom part of the foot not subjected to the light treatment may not have the secondary coating). The ECD could be modified by cutting the end of the ECD that encompasses any of the digits that have nails to be treated. The ECD covering digits of nails not receiving treatment would remain intact.

DCD—Digit Covering Device—The second piece of the system in the preferred embodiment is similar to a finger cot but comes in a variety of sizes permitting the correct size to be selected for the digit being treated. This piece is designed to provide comfort during treatment, total blocking of light during treatment, and can also be used to provide ventilation and cooling as necessary. The DCD will be fitted to the subject based on the size of the digit being treated. This piece has a cutout on top that will permit the nail to be exposed during treatment. This piece covers most of the skin around the nail to be treated. However, in the preferred embodiment the DCD is designed to be used with a third piece which is an adhesive masking system that covers the small amount of skin that remains unmasked once the digit covering unit is in place. In the preferred embodiment the digit covering unit will comprise something similar to a finger cot with a (latex) rubber exterior and lined interior (maybe cotton). The DCD shall have a positive means to permit it to stay in place (interior adhesive, adhesive strip placed around its circumference and the skin, etc.).

SCD—Skin Covering Device—the third piece of the system in the preferred embodiment is a skin masking unit which permits the masking of small amount of skin exposed between the DCD and the nail to be treated. This unit comprise a material that can shield the skin from the light used during treatment. In the preferred embodiment the SCD is a thick (approximately ⅛—in or more) tape or bandage that can block light, is readily conformable, and can adhere well to the skin being masked. In the preferred embodiment the ECD would be similar to microfoam tape as manufactured by 3M.

Description of applying a preferred embodiment of the system—first select and apply the DCD. Secondly, apply the SCD around the nail to be treated to minimize the amount of skin exposed to treatment. Thirdly, select and modify as necessary the ECD and put it on the extremity that has the nail to be treated. Applying in this sequence permits part of the SCD to be in contact with the DCD so that part of the adhesive adheres there and less adhesive is in contact with the skin. Application in this order facilitates the removal of the SCD since it can be removed at one time with the DCD and this makes its removal quicker, less painful (since there is less adhesive in contact with the skin and therefore also less chance of the adhesive adhering to hair), and also minimizes possibility of an allergic reaction to the adhesive. This order of application is specifically claimed as a preferred embodiment of this invention.

FIGS. 14 shows pictorial application instructions of a two part system for treating nails on the hand or the foot. These instructions may be supplemented with written instructions or the written instructions could be used instead of the pictorial instructions.

### Additional Preferred Embodiment

Alternately, in another preferred embodiment the DCD could be applied first, the ECD second, and the SCD third. This would have the advantage of preventing the application of the ECD from disturbing the SCD and also facilitate minor repositioning of the DCD before the SCD is applied. This order of application is also specifically envisioned as a preferred alternative of this invention.

### Additional Preferred Embodiment

Another preferred embodiment would be to change the sequence of application so that the ECD was first applied, then the DCD would be applied over it, and finally the SCD would be applied. The advantage of this sequence is that it may be possible to remove all the units at once when done. It would also minimize skin contact with the DCD and the SCD. However, it may also be more difficult to apply the DCD (and subsequently the SCD) with the ECD already in place.

### Additional Preferred Embodiment

Alternately, another preferred embodiment is to first apply the SCD, then apply the DCD over it, and finally apply the ECD. This latter sequence permits the skin to be covered first by a SCD that is wide enough to cover approximately ¼-in all around the nail (or ⅜ to ½-inch around large nail). A wide SCD permits the use of a smaller number of DCD sizes since the holes can be considerably larger than the nail opening itself This sequence of application may also make it less likely that the SCD will shift; however, it also can make the repositioning of the SCD more difficult.

A preferred embodiment can be applied such that the ECD, the DCD, and the SCD can be applied in different order to best achieve the desired results.

A preferred embodiment can be applied similarly to the hands if a fingernail is being treated with the ECD being conformable to the hand instead of the foot.

A preferred embodiment can be applied to skin in other parts of the body besides the extremities and in such case the ECD would not be required.

### Additional Preferred Embodiment

An additional preferred embodiment is a special modification of the SCD which uses a transparent covering to first be placed over the area being treated and an outline of the area to be treated to be traced upon the transparent covering. The transparent covering may have grid lines or other indicia to aid in marking, cutting or identifying the area to be cut. The transparent covering can then be transferred to the top of the treatment shield or covering. A knife, scissors, or similar item can then be used to cut out the opening. The treatment covering can then be applied to cover the areas surrounding the area to be treated. Alternatively other similar methods could be used such as taking a photo and scanning it and then using it to make an opening of the right size to permit treatment of an infection while masking the surrounding area. The SCD can be used alone if the area is not covering an extremity.

### DESCRIPTION OF FURTHER PREFERRED EMBODIMENTS

### Further Preferred Embodiments

In another preferred embodiment one or more of the parts—the ECD, DCD, and SCD may be combined.

In another preferred embodiment the DCD can be made long so that it can be trimmed (short for small digits like small toes or left long to treat longer digits such as a small fingernail).

In another preferred embodiment the ECD could be made out of paper or foam. While a toe sock could be used, a less expensive temporary cutout could work just as well (especially in conjunction with a properly designed DCD and SCD).

In another preferred embodiment translucent material that is impervious to UV light (99% rejection of UVA and 99.9% rejection of UVB and UVC) could be used. This would help with fitting of the units (especially the ECD and getting the toes or fingers in).

In another preferred embodiment the DCD may not have a nail opening precut but may instead be cut to better match the particular nail opening (using a device similar to a hole punch that can be fitted with dies that will cut out different size openings or other means that accomplish this end). The DCD may be installed over a foot or hand shaped block or mandrel while the punch or die is used to cut through one layer of the DCD to prevent the bottom (i.e., the surface underneath the foot of the user when the DCD is installed on a patient) of the DCD from being cut. Alternatively, the punch could cut through the top and bottom layers of the DCD, since the bottom layer is not typically directly adjacent an area being treated. The DCD could also have a small transparent material that you could ‘trace the nail’ with and then use to cut the opening better as described above.

The system could be sold as a single pack that would contain one digit covering device and would have a clear plastic disposable nail template that could be used to determine the best size to cut the opening—each cutout having a number or other indicia associated with it. When the correct cutout size is selected, a corresponding number could then be dialed into the cutting device and it would cut an opening the desired size corresponding to the one selected. The cutting device could have dies with numbers on them and when the right number was selected the corresponding numbered die could be used.

In another preferred embodiment the means of covering can be loose fitting or can conform the part to the body being covered or can be a combination of these two or may have various portions that are form fitting and various portions that are loose.

In another preferred embodiment the conformation material may include items such as covering made for digits (such as toe socks, gloves, etc.) specially modified to make it easy to expose areas to be treated (precut openings that are easily exposed or a covering for a finger/nail—cylinder with end covered—that an opening can be cut into by use of a punch similar to a hole punch but with templates that can be interchanged to make sure they cut the right size openings.

In another preferred embodiment coverings can be on a flat sheet (tacky on the back with slick paper to remove to expose tackiness). The proper cutout is then selected (it is selected by putting the dies or templates of the dies over the area to be treated and once finding the right one it is then used to punch out the correct opening) and used to cut the opening. Then the covering is applied (to the toe or finger, etc.).

Another preferred embodiment is an appliqué that is transparent in visible wavelengths and can be cut away to expose areas to be treated (use a ‘LASIK’ type knife to effectively cut the appliqué without hurting the skin. An appliqué that is transparent that can be applied to skin and the opening traced on it. The appliqué is then removed and put over the mask that is to be used and an opening is cut out of both. Once the mask has the correct opening cut it can be applied to the area to be treated. The mask may have special properties such a optical opacity at certain wavelengths, may be make of a material that prevents slippage (such as a sticky or tacky surface), may reject heat generated (by reflection, etc.), may have secondary uses such a being able to cool an area with embedded thermoelectric fibers that do microrefridgeration, etc.

In another preferred embodiment the covering could be used in an area that is not an extremity and has only one part, such as the SCD, that is sufficient to cover the skin adjacent area being treated. There may be one or more openings for treatment of specific areas.

In a preferred embodiment each one of the ECD, DCD, and SCD may be disposable or reusable.

In a preferred embodiment the ECD, DCD, and SCD may be made of various materials including but not limited to Natural Rubber, Synthetic Rubber, Butadiene, SBR, Butyl, EP Rubber, Neoprene, Nitrile, Silicone, and Urethane. The material used for one part may or may not be used for another part.

In a preferred embodiment the thickness of each item may be between approximately 0.4 mil and 250 mils.

In a preferred embodiment the finish of each item may be smooth, granular, diamond pattern, raised, crepe, or a similar finish.

In a preferred embodiment the interior finish of each item may be plain, cotton based, kitted liner, chlorinated, hypoallergenic, powder free, powdered, flocked, or a similar finish. In a variant of the interior finish the powdered finish could have an antibiotic material such as 2% Miconazole Nitrate with an inert mixture of Corn Starch, Corn Starch, Polymer, Fragrance, Talc, and similar material.

In a preferred embodiment the different properties of the materials used such as durometer, elongation, tear resistance, and biocompatibility are taken into account to optimize the use of each item.

In a preferred embodiment each item can be made by dipping, flat sheets (welded or similarly joined), molding, and combinations of these.

In other preferred embodiments coverings can be impervious to medications that are used before or after treatment. The covering can be used before or after treatment with medications that can enhance the efficacy of treatment. For example, a covering could be applied treatment. Once phototherapy treatment is finished it can be left in place to protect skin from treatment with a topical application. Thus, a sorbent pad with antifungal medication could be applied after treatment and left in place for a day. Each day the pad could be replaced. If the pad and covering were constructed in a certain manner they would be relatively resistant to water and could thus be worn for several days even if a shower is taken. Conversely, use of the covering that is left in place for a day and is not waterproof could be a way to keep people from getting the area wet if that is undesirable.

In other preferred embodiments a different sequence of covering can be used. If the DCD and SCD is applied before the ECD then they must have means to stay in place and not shift, that is they may need some way to fix the end farthest from the nail with a rubber or elastic ring, adhesive that is uncovered by removing a strip, etc. If applied before the ECD, then it may be necessary to cut off the end of each digit covering on the nail to be treated. Also, once the ECD is removed, the SCD will stay in place and be easier to use for medication follow-up. If the covering is applied after a sock is in place, it will require that the sock have precut openings and it will be more difficult to use after treatment.

In another preferred embodiment, cooling tubes could be part of the ECD, DCD, and/or SCD if significant heat is generated during treatment. Or an air plenum could be used to keep the toe cool.

In another preferred embodiment the ECD, DCD, and/or SCD have markings on them to ensure they are properly aligned with each other and with the treatment device. For example, the EDC may have an ‘insertion line’ marked on the foot glove to make sure the foot is properly inserted into the treatment device the proper distance and/or in proper alignment.

In another preferred embodiment the ECD, DCD, and or SCD may have a quality that can be detected by the treatment device and that interlocks with the treatment device to permit treatment only if the ECD, DCD, or SCD is present. For example, the ECD could have an RFID chip embedded in it which the treatment device would detect prior to providing treatment. Another example would be that the SCD may have a type of fluorescence that the treatment device can detect and that the treatment device will only run when it detects this.

### Further Alternative Embodiments

Coverings can include attachments or structures that are integral to the phototherapy device that limit the area of light that radiates from the device. These attachments can be in contact with the skin. Also, these attachments can be disposable or can be reusable.

Coverings to provide blocking superior to sunscreen. Also blocking uses materials that are contact with the skin that are non-irritating with the material that blocks well on the other side away from the skin. Therefore, it may be necessary to have a covering that can block more than three orders of magnitude of UV light. An alternative embodiment would be to apply a sun block (i.e., UV block) underneath the ECD, DCD, and SCD to enhance the blockage of UV light.

Although the descriptions above contain many specificities, these should not be construed as limiting the scope of the invention but merely as providing illustrations of some of the presently preferred embodiments of this invention.

We claim:

1. A covering system for selectively exposing and masking portions of a patient for use during therapeutic treatment, comprising at least two devices selected from the group of:
a. an extremity covering device for masking a large area of the patient from said therapeutic treatment, said extremity covering device having a first opening for receiving a hand or foot of a patient and a second opening for exposing a first area of the patient;
b. a digit covering device for masking a portion of said first area of the patient from said therapeutic treatment and for exposing a second area of the patient smaller than said first area; and
c. a skin covering device for masking a portion of said second area of the patient from said phototherapy treatment and for exposing a third area of the patient smaller than said third area of the patient.

2. A covering system according to claim 1, wherein one of the group of the extremity covering device, the digit covering device and the skin covering device is formed of a stretchable material, wherein said material when stretched continues to mask a portion of the patient from said phototherapy treatment.

3. A covering system according to claim 1, wherein said extremity covering device further includes a flange section having an elastomeric band for securing on a respective flange of a phototherapy treatment device to prevent light from escaping from the phototherapy treatment device past said extremity covering device during therapeutic treatment.

4. A covering system according to claim 1, wherein said extremity covering device further includes a flange section for being received within a phototherapy treatment device to prevent light from escaping from the phototherapy treatment device past said extremity covering device and to secure the extremity covering device to the phototherapy device.

5. A covering system according to claim 1, wherein one of the group of the extremity covering device, the digit covering device and the skin covering device includes indicia on the device to provide cutting guides.

6. A covering system according to claim 1, wherein the indicia is a template.

7. An elastomeric covering for a foot having an opening defined for each toe of the foot and having a bottom portion that covers a substantial part of the sole of the foot, wherein said elastomeric covering covers at least up to the ball of the foot, substantially all of the sole, and all of the sole up to and including the heel.

8. A system for covering the area surrounding a nail to be treated by phototherapy comprising:
an adhesive strip comprising an arcuate shaped material having a base and two opposing legs spaced apart from each other.

9. A system according to claim 8, wherein the adhesive strip is comprises a material having an approximate shape selected from the group of an arc, a semicircle, a ‘U’ shape, and a circle.

10. A system according to claim 8, wherein the adhesive strip is opaque to ultraviolet light.

11. A system according to claim 8, wherein the adhesive strip comprises an ultraviolet light blocking material.

12. A method of covering at least one nail on the hand or foot of a patient comprising:
providing an extremity covering device formed of a first material defining a interior space covering substantially covering one surface of the hand or foot of the patient,
forming an opening in the surface of said extremity covering device to expose at least one nail to be treated; wherein said opening is constructed by one of forming said opening during original manufacturing, removing a portion of said covering device adjacent said nail after determining an amount of material to remove to expose at least one nail being treated; using a die to cut out a preselected size of material from the device; and removing a scored portion of material prior to treating the patient, and providing a skin covering device formed of a second material defining a interior space masking an area of skin adjacent said opening between the nail or nails to be treated and the extremity covering device.

13. The method of covering according to claim 12, wherein at least one of said extremity covering device first material and said skin covering device second material is formed of an opaque material filtering at least 99% of any light having a wavelength of less than 400 nm from passing through the material to the respective interior space therein.

14. The method of covering according to claim 12, wherein at least one of said extremity covering device first material and said skin covering device second material is formed of an opaque material blocking at least 99.5% of any light having a wavelength of 240 nm to 400 nm from passing through the material to the respective interior space therein.

15. The method of covering according to claim 12, wherein at least one of said extremity covering device first material and said skin covering device second material is formed from a material rated to block at least 99.5% of any light having a wavelength of 240 nm to 400 nm from passing through the material to the respective interior space therein.

16. The method of covering according to claim 12, wherein at least one of said extremity covering device first material and said skin covering device second material is tested to verify that the material is capable of blocking at least 99.5% of any light having a wavelength of 240 nm to 400 nm from passing through the material to the respective interior space therein.

17. The method of covering of according to claim 12, wherein said coverings having been determined to filter 99% of UV light from penetrating the covering as ascertained by using material and construction that has been determined to filter 99% of said UV light by selecting from a group composed of: calculating the necessary characteristics of said coverings to filter said light, selecting materials rated to filter said light, individual testing of materials to substantiate their ability to filter 99% of UV light, statistically significant sample to ensure the materials have the ability to filter 99% of UV light, or any combination thereof.

18. The method of covering according to claim 17, wherein at least one of said extremity covering device first material and said skin covering device second material is formed of an elastomeric.

19. A covering for at least one toe of a patient having at least one nail receiving therapeutic treatment, the covering comprising:
an elastomeric material including a plurality of extrusions extending from a foot covering portion;
each of said extrusions enclosing at least one toe;
said foot covering portion covering at least one inch of the foot beyond the toes;
said elastomeric material preventing at least 99% of any light having a wavelength less than 400 nm from passing through the elastomeric material to an inside portion of the covering.

20. The method of covering according to claim 19, wherein the covering has tabs on at least one extrusion for easily removing a portion of the covering to expose at least one nail to be treated.

21. The method of covering claim 19, wherein the covering interfaces with a phototherapy device providing treatment.

22. The method of covering according to claim 21, wherein the covering has a flange that mates with a respective flange on the phototherapy device.

23. The method of covering according to claim 21, wherein a portion of the covering extends outward from the foot to mate with the treatment device opening to substantially cover an opening on the phototherapy device to prevent light from escaping from said opening past said covering.

24. The method of covering according to claim 19, wherein said covering material is chosen from materials rated to block at least 99.5% of any light having a wavelength less than 400 nm from passing through the material to the respective interior space therein.

25. The method of covering according to claim 19, wherein said coverings having been determined to filter 99% of UV light as ascertained by using material and construction that has been calculated, rated, or tested in order to substantiate its ability to filter of 99% of UV light.

26. A covering for masking an area of skin adjacent to a nail receiving therapeutic treatments comprising an arcuate adhesive strip with a shape selected from a group consisting of an arc, a semicircle, a ‘U’ shape, a ‘horseshoe’ shape, a complex curve, and a combination thereof.

27. The covering of claim 26, further comprising a backing layer to protect the adhesive prior to deployment.

28. The method of covering claim 26, wherein the covering is made from a material that is an opaque material f at least 99% of any light having a wavelength of less than 400 nm from passing through the material to an underside thereof.

29. The method of covering claim 26, wherein the covering is made from a material that is an opaque material blocking at least 99.5% of any light having a wavelength of 240 nm to 400 nm from passing through the material to an underside thereof.
